(19) United States
(12) Patent Application Publication
(10) Pub. No.: US 2004/0106127 A1
(43) Pub. Date: Jun. 3, 2004

(54) Novel gene and protein encoded by the gene

(75) Inventors: Takahiro Nagase, Kisarazu-shi (JP); Daisuke Nakajima, Kisarazu-shi (JP); Osamu Ohara, Kisarazu-shi (JP); Reiko Kikuno, Kisarazu-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: KAZUSA DNA RESEARCH INSTITUTE FOUNDATION, Kisarazu-shi (JP); ProteinExpress Co. Ltd., Choshi-shi (JP)

(21) Appl. No.: 10/601,807

(22) Filed: Jun. 24, 2003

(30) Foreign Application Priority Data
Jul. 15, 2002 (JP) 2002-205915
Jun. 12, 2003 (JP) 2003-167684

(51) Int. Cl. C12Q 1/68; C07H 21/04; C12N 9/00; C07K 14/47; C07K 14/705

(52) U.S. Cl. 435/6; 435/69.1; 435/183; 435/320.1; 435/325; 530/350; 536/23.2

(57) ABSTRACT

Novel DNAs containing the regions which encodes proteins have been directly cloned from cDNA libraries derived from the human adult whole brain and the human embryonic whole brain, the nucleotide sequences thereof have been determined, and their functions have been identified. The present invention provides DNA which comprises the nucleotide sequence encoding the following polypeptide (a) or (b):

(a) a polypeptide comprising an amino acid sequence which is identical or substantially identical to an amino acid sequence represented by any one of SEQ ID NOA: 1 to 3;

(b) a polypeptide comprising an amino acid sequence derived from the amino acid sequence represented by any one of SEQ ID NOS: 1 to 3 by deletion, substitution or addition of a section of amino acids, and having biological activity which is substantially the same characteristic with the function of the polypeptide of (a); a recombinant polypeptide, which is encoded by the above DNA; and a protein containing the polypeptide.

### TECHNICAL FIELD

The present invention relates to DNA and a gene containing the DNA, and a recombinant polypeptide encoded by the DNA and a novel recombinant protein containing the polypeptide.

### BACKGROUND ART

An enormous amount of information on the nucleotide sequence of the human genome has been obtained by large-scale sequencing in the Human Genome Project and analysis of the information is continuing on a daily basis.

The ultimate goal of the Human Genome Project is not just simple determination of the entire nucleotide sequence of the genome, but also the elucidation of various human life phenomena based on the structural information, that is the nucleotide sequence information of DNA.

Only limited regions of the human genome sequence encode proteins. Currently, the coding regions are predicted by the neural network or an information science technique, called the Hidden Markov Model. However, these models' predictive abilities are not yet sufficiently reliable.

### DISCLOSURE OF THE INVENTION

For the purpose of finding novel genes, we have completed the present invention by succeeding in directly cloning novel DNAs comprising regions that encode proteins from cDNA libraries derived from the human adult whole brain and the human embryonic whole brain, and determining the nucleotide sequences thereof.

In a first embodiment, the present invention relates to DNA comprising a nucleotide sequence encoding the following (a) or (b):

(a) a polypeptide consisting of an amino acid sequence which is identical or substantially identical to an amino acid sequence represented by any one of SEQ ID NOS: 1 to 3;

(b) a polypeptide consisting of an amino acid sequence derived from an amino acid sequence represented by any one of SEQ ID NOS: 1 to 3 by deletion, substitution or addition of a section of amino acid(s), and having biological activity which is substantially the same characteristic with the function of the polypeptide of (a). Examples of such DNA include, but are not limited to, DNAs comprising the nucleotide sequences of SEQ ID NOS: 1 to 3.

In a second embodiment, the present invention further relates to a DNA hybridizing to the DNA of the first embodiment of the present invention under stringent conditions, and encoding a polypeptide having biological activity which is substantially the same characteristic with the function of the polypeptide of (a) above.

Hereinafter, the DNAs of the first and the second embodiments of the present invention are together referred to as “the DNA of the present invention”. Further, the present invention also relates to antisense DNA comprising a nucleotide sequence which is substantially complementary to the DNA of the present invention.

In a third embodiment, the present invention relates to a gene construct containing the DNA of the present invention. The term “gene construct” in the present specification refers to every artificially-engineered gene. Examples of the gene construct includes, but are not limited to, a vector containing the DNA of the present invention or the antisense DNA of the DNA of the present invention, and an expression vector of the DNA of the present invention.

In a fourth embodiment, the present invention relates to the following (a) or (b):

(a) a polypeptide, consisting of an amino acid sequence which is identical or substantially identical to an amino acid sequence represented by any one of SEQ ID NOS: 1 to 3;

(b) a polypeptide, consisting of an amino acid sequence derived from the amino acid sequence represented by any one of SEQ ID NOS: 1 to 3 by deletion, substitution or addition of a section of amino acids, and having biological activity which is substantially the same characteristic with the function of the polypeptide of (a).

In a fifth embodiment, the present invention relates to a recombinant polypeptide encoded by the gene construct of the third embodiment of the present invention.

Hereinafter, the above polypeptides are together also referred to as “the polypeptide of the present invention.” The term “polypeptide” in the present specification refers to “polymers of amino acids having every molecular weight.” The present invention also relates to a recombinant protein containing the polypeptide of the present invention. As defined above, in the present specification the term “polypeptide” is not to be limited by molecular weight, and therefore the term “the polypeptide of the present invention” also includes a recombinant protein containing the polypeptide of the present invention.

In a sixth embodiment, the present invention relates to an antibody against the polypeptide of the present invention.

In a seventh embodiment, the present invention relates to a DNA chip on which the DNAs of the present invention are arrayed.

In an eighth embodiment, the present invention relates to a polypeptide chip on which the polypeptides of the present invention are arrayed.

In a ninth embodiment, the present invention relates to an antibody chip on which the antibodies of the sixth embodiment of the present invention are arrayed.

Table 1 shows the names of clones having the DNA of the present invention, lengths of the polypeptide of the present invention and their putative functions.

The DNAs of the present invention are identified by determining the nucleotide sequences after isolating them as cDNA fragments from cDNA libraries that we have prepared using as a starter material the commercially available (Clontech) mRNA of the human adult whole brain and the human embryonic whole brain.

Specifically, clones are randomly isolated from cDNA libraries derived from the human adult whole brain and the human embryonic whole brain prepared according to the method of Ohara et al. (DNA Research 4:53 59 (1997)).

Both termini of the nucleotide sequences are determined for a clone. Using the thus obtained terminal nucleotide sequences as queries, homology search was run on known gene database. As a result, the clones shown to be new were related to human genome sequences with 5′ and 3′ termini sequences of the cDNA followed by cDNA full-length determination for the clones confirmed that they contain unknown long chain gene in the region interleaved in these termini.

Further, the entire region of a human-derived gene containing the DNA of the present invention can also be prepared by a PCR method, such as RACE, while exercising proper care so as not to cause short fragments or any artificial mistakes in obtained sequences.

Furthermore, the present invention provides a recombinant vector which comprises the DNA of the present invention or a gene construct containing the DNA of the present invention; a transformant retaining the recombinant vector; a method for producing the polypeptide of the present invention or a recombinant protein containing the polypeptide, or salts thereof, which is characterized by culturing the transformant, producing and accumulating the polypeptide of the present invention or the recombinant protein containing the polypeptides, and collecting these products; and the thus produced polypeptide of the present invention or the recombinant protein containing the polypeptide, or salts thereof.

The present invention also relates to a pharmaceutical preparation comprising the DNA of the present invention or the gene construct; a pharmaceutical preparation comprising a polynucleotide (DNA) comprising a nucleotide sequence which encodes the polypeptide of the present invention or a partial polypeptide thereof, or a recombinant protein containing the polypeptides, an antisense nucleotide comprising a nucleotide sequence substantially complementary to the nucleotide sequence which encodes the polypeptide of the present invention or a partial polypeptide thereof, or a recombinant protein containing the polypeptides; a pharmaceutical preparation comprising the polynucleotide of the present invention and the antisense nucleotide; and a pharmaceutical preparation comprising the polypeptide of the present invention or a partial polypeptide thereof and a recombinant protein containing the polypeptides.

The present invention further relates to a DNA chip, a peptide chip and an antibody chip that are prepared by arraying the DNAs of the present invention, the polypeptides of the present invention and the antibodies against the polypeptide of the present invention, respectively.

The present invention further relates to an antibody against the polypeptide of the present invention or a partial polypeptide thereof or a recombinant protein containing the polypeptides, or against salts thereof and a method for screening a substance which specifically interacts with the polypeptide of the present invention by using the polypeptide of the present invention, a partial polypeptide thereof or a recombinant protein containing the polypeptides, or salts thereof, or antibodies against these substances; a kit for screening; and the substance (compound) itself which is identified by the screening method.

Any DNA can be used as the DNA of the present invention, so far as it comprises a nucleotide sequence encoding the above-mentioned polypeptide of the present invention. Further, the DNA of the present invention may be cDNA identified and isolated from cDNA libraries or the like derived from the human brain, from cells or tissues other than brain, such as the heart, lung, liver, spleen, kidney and testicle, or synthetic DNA.

A vector used for constructing libraries may be a bacteriophage, a plasmid, a cosmid, or a phagemid. In addition, using total RNA fractions or mRNA fractions prepared from the above cells or tissues, amplification can be performed directly by a reverse transcriptase-polymerase chain reaction (hereafter, abbreviated as “RT-PCR method”.).

Any antisense DNA may be used as an antisense oligonucleotide (DNA) having a nucleotide sequence substantially complementary to the DNA that encodes the polypeptide of the present invention or a partial polypeptide thereof, so far as it comprises a nucleotide sequence substantially complementary to the nucleotide sequence of the DNA, and is capable of inhibiting the expression of the DNA. A substantially complementary sequence is, for example, a nucleotide sequence having preferably about 90% or more, more preferably about 95% or more, and most preferably 100% homology with the full-length or partial nucleotide sequence of the nucleotide sequence complementary to the DNA of the present invention. The antisense DNA of the present invention includes a nucleic acid sequence (RNA or DNA modified) having a similar function to that of the antisense DNA. These antisense DNAs can be produced using a known DNA synthesizer or the like.

The term “an amino acid sequence substantially identical to an amino acid sequence represented by any one of SEQ ID NOS: 1 to 3” refers to an amino acid sequence having on the overall average about 70% or more, preferably about 80% or more, further preferably about 90% or more, and particularly preferably about 95% or more homology with each of all the amino acid sequence represented by any one of SEQ ID NOS: 1 to 3.

An example of a polypeptide consisting of an amino acid sequence substantially identical to amino acid sequence represented by any one of SEQ ID NOS: 1 to 3 of the present invention is a polypeptide having the above homology with the amino acid sequence represented by each of the above SEQ ID NOS, and having biological activity (function) which is substantially the same characteristic with the function of the polypeptide comprising the amino acid sequence represented by each SEQ ID NOS. The term “substantially the same characteristic” refers to the activity (function) having the same characteristics.

Further, the polypeptide of the present invention also includes, for example, a polypeptide consisting of an amino acid sequence derived from an amino acid sequence represented by any one of SEQ ID NOS: 1 to 3 by deletion, substitution or addition of a section of amino acids (preferably about 1 to 20, more preferably about 1 to 10, and further preferably several amino acids) or by a combination of these, and having biological activity (function) which is substantially the same characteristic with the function of a polypeptide comprising an amino acid sequence represented by any one of SEQ ID NOS: 1 to 3.

The polypeptide consisting of an amino acid sequence which is substantially identical to the above amino acid sequence represented by any one of SEQ ID NOS: 1 to 3, or the polypeptide comprising an amino acid sequence derived from the above amino acid sequence by deletion, substitution or addition of a section of the amino acids can be easily produced by, for example, an appropriate combination of methods known by a person skilled in the art, such as site-directed mutagenesis, homologous recombination of genes, primer elongation and PCR.

For the polypeptide to have biological activity which is substantially the same characteristics, a possible method is substitution between homologous amino acids (polar or nonpolar amino acids, hydrophobic or hydrophilic amino acids, positively or negatively charged amino acids, aromatic amino acids and the like) among amino acids composing the polypeptide. To maintain biological activity that is substantially the same characteristics, it is preferred to retain amino acids within functional domains contained in each polypeptide of the present invention.

Further, the DNA of the present invention includes DNA comprising a nucleotide sequence encoding an amino acid sequence represented by any one of SEQ ID NOS: 1 to 3, and a DNA hybridizing to the DNA under stringent conditions, and encoding a polypeptide having a biological activity (function) which is the same characteristic with the function of a polypeptide consisting of an amino acid sequence represented by each of the sequences.

Under such conditions, examples of DNA capable of hybridizing to DNA comprising a nucleotide sequence, encoding an amino acid sequence, presented by each of the nucleotide sequences of SEQ ID NOS: 1 to 3 include DNA comprising a nucleotide sequence having on the overall average about 80% or more, preferably about 90% or more, more preferably about 95% or more homology with each one of all the nucleotide sequence of the DNAs.

Hybridization can be performed by a method known in the art or a method according to any known methods, such as a method described in Current Protocols in Molecular Biology (edited by Frederick M. Ausubel et al., 1987). When a commercially available library is used, hybridization can also be performed by the method described in the attached instructions.

The term “stringent conditions” means, for example, conditions that allow hybridizing to the DNA probe of the present invention by southern blot hybridization under conditions that involve hybridization in an 7% SDS solution containing 1 mM sodium EDTA and 0.5 M dibasic sodium phosphate (pH 7.2) at 65° C., and washing membranes in a 1% SDS solution containing 1 mM sodium EDTA and 40 mM dibasic sodium phosphate (pH 7.2) at 65° C. The same stringency can also be achieved by conditions other than the above conditions.

To clone the DNA of the present invention, amplification is performed by a PCR method using a synthetic DNA primer having an appropriate nucleotide sequence of a part of the polypeptide of the present invention or the like, or the DNA can be selected by hybridization of DNA incorporated in an appropriate vector with DNA labeled using a DNA fragment of synthetic DNA which encodes a section or the full-length region of the polypeptide of the present invention.

Hybridization can be performed according to, for example, the above-described method in “Current Protocols in Molecular Biology” (edited by Frederick M. Ausubel et al., 1987). In addition, when commercially available libraries are used, hybridization can be performed according to the method described in the attached instructions.

Cloned DNA encoding a polypeptide can be used intact, or can be used after digestion with restriction enzymes if necessary, or after addition of linkers thereto, depending on the purpose. The DNA may contain ATG as a translation initiating codon at the 5′ terminal side, or TAA, TGA or TAG as a translation termination codon at the 3′ terminal side. These translation initiating and termination codons may be added using an appropriate synthetic DNA adaptor.

An expression vector for the polypeptide of the present invention can be produced according to any method known in the technical field. For example, the vector can be produced by (1) cleaving a DNA fragment containing the DNA of the present invention or a gene having the DNA of the present invention, and (2) ligating the DNA fragment downstream of a promoter in an appropriate expression vector.

Examples of vectors that can be used herein include plasmids derived from Escherichia coli; (for example, pBR322, pBR325, pUC18, pUC118), plamids derived from Bacillus subtilis (for example, pUB110, pTP5, pC194), plamids derived from yeast (or example, pSH19, pSH15), bacteriophages, such as λ phages, and animal viruses, such as retrovirus, vaccinia virus, baculovirus and the like.

Any promoter can be used in the present invention, so far as it is appropriate for a host to be used for gene expression. Preferred examples of promoters include, when the host is Escherichia coli, trp promoters, lac promoters, reca promoters, λPL promoters and lpp promoters; when the host is Bacillus subtilis, SPO1 promoters, SPO2 promoters and penP promoters; and the host is yeast, PHO5 promoters, PGK promoters, GAP promoters and ADH promoters. When animal cells are used as promoters, examples of promoters include SRα promoters, SV40 promoters, LTR promoters, CMV promoters and HSV-TK promoters.

In addition to the above substances, an enhancer, splicing signal, polyA addition signal, a selection marker, SV40 replication origin and the like that are known in the technical field can be added to the expression vector, if desired. Further, if necessary, a protein encoded by the DNA of the present invention can be expressed as a fusion protein with another protein (for example, glutathione S transferase and protein A). Such a fusion protein can be cleaved with appropriate protease and then separated into each protein.

Examples of host cells that are used herein include bacteria of the genus Escherichia or the genus Bacillus, yeast, insect cells, and animal cells.

Specific examples of bacteria of the genus Escherichia that are used herein include Escherichia coli K12/DH1 (Proc. Natl. Acad. Sci USA, 60:160 (1968)), JM103 (Nucleic Acids Research, 9:309 (1981)), JA221 (Journal of Molecular Biology, 120:517 (1978)), and HB101 (Journal of Molecular Biology, 41:459 (1969)).

Examples of bacteria of the genus Bacillus that are used herein include Bacillus subtilis MI114 (Gene, 24:255 (1983)) and 207-21 (Journal of Biochemistry, 95:87 (1984)).

Example of yeast that are used herein include Saccaromyces, such as Saccaromyces cerevisiae AH22, AH22R-, NA87-11A, DKD-5D, 20B-12; Shizosaccaromyces pombe NCYC1913, NCYC2036; and Pichia pastoris.

Examples of animal cells that are used herein include monkey cells, such as COS-7 and Vero, Chinese hamster ovary cells, such as CHO (hereinafter, abbreviated as CHO cells), dhfr gene-deficient CHO cells, mouse L cells, mouse AtT-20, mouse myeloma cells, rat GH3, and human FL cells.

These host cells can be transformed according to a method known in the technical field. For example, transformation can be performed by referring to Proc. Natl. Acad. Sci. USA, 69:2110 (1972); Gene, 17:107 (1982); Molecular & General Genetics, 168:111 (1979); Methods in Enzymology, 194:182 (1991); Proc. Natl. Acad. Sci. USA, 75:1929 (1978); A supplementary volume 8 of Cell Technology, New Experimental Protocols in Cell Technology, 263-267 (1995) (issued by Shujunsha); and Virology, 52:456 (1973)).

The thus obtained transformant, which has been transformed with an expression vector containing the DNA of the present invention or a gene containing the DNA of the present invention, can be cultured according to a method known in the technical field.

For example, when hosts are bacteria of the genus Escherichiae, culturing is performed normally at about 15° C. to 43° C. for about 3 to 24 hours, and if necessary, aeration and agitation may be performed. When hosts are bacteria of the genus of Bacillus, culturing is performed normally at about 30° C. to 40° C. for about 6 to 24 hours, and if necessary, aeration and agitation may be performed.

A transformant whose host is yeast is normally cultured using media adjusted to have pH of approximately 5 to 8, at about 20° C. to 35° C. for about 24 to 72 hours, and if necessary, aeration and agitation may be performed.

A transformant whose host is an animal cell is normally cultured using media adjusted to have pH of about 6 to 8, at about 30° C. to 40° C. for about 15 to 60 hours, and if necessary, aeration and agitation may be performed.

To isolate and purify the polypeptide or the protein of the present invention from the above culture product, for example, bacteria or cells are collected by a known method after culturing, suspended in an appropriate buffer, disrupted by ultrasonication, lysozyme and/or freezing and thawing, and then centrifuged or filtered, thereby obtaining a crude protein extract. The buffer may contain a protein-denaturing agent, such as urea or guanidine hydrochloride, or a surfactant, such as Triton X-100 (trademark). When the protein is secreted in a culture solution, bacteria or cells are separated after culturing from the supernatant by a known method, thereby collecting the supernatant. The thus obtained culture supernatant or the protein contained in an extract can be purified by an appropriate combination of known isolation and purification methods.

The thus obtained polypeptide of the present invention can be converted to a salt by a known method or a method according to the known method. Conversely, when the polypeptide is obtained as a salt, it can be converted to an educt or another salt by a known method or a method according to the known method. Further before or after purification, the protein produced by a recombinant can be freely modified or removed partially its polypeptide by allowing an appropriate protein modification enzyme, such as trypsin and chymotrypsin, to act on the protein.

The presence of the polypeptide of the present invention or its salt can be measured by various binding assays and enzyme immunoassay using a specific antibody.

The C-terminus of the polypeptide of the present invention is normally a carboxyl group (—COOH) or a carboxylate (—COO—), and the C terminus may be an amide (—CONH₂) or ester (—COOR). Here, examples of R in ester that are used herein include a C1-6 alkyl group, such as methyl, ethyl, n-propyl, isopropyl or n-butyl; a C3-8 cycloalkyl group, such as cyclopentyl or cyclohexyl; a C6-12 aryl group, such as phenyl or α-naphthyl; a phenyl-C1-2 alkyl group, such as benzyl or phenethyl; and a C7-14 aralkyl group, such as an α-naphthyl-1-2 alkyl group, e.g., α-naphthyl methyl. Further, pivaloyl-oxymethyl ester being generally used as oral administration may also be used.

When the polypeptide of the present invention has a carboxyl group (or carboxylate) other than at the C-terminus, the polypeptide of the present invention encompasses such a polypeptide wherein carboxyl group is amidated or esterified. An example of ester that is used in this case is the above-mentioned ester at the C-terminus. Moreover the polypeptide of the present invention also encompasses a polypeptide wherein an amino group of a methionine residue at the N-terminus is protected with a protecting group (for example, a C1-6 acyl group, such as a formyl group or an acetyl group); a polypeptide wherein a glutamic acid residue at the N-terminus which is generated by in vivo cleavage is pyroglutamated; a polypeptide wherein OH, COOH, NH₂, SH and the like on the side chain of intramolecular amino acids are protected with appropriate protecting groups (for example, a C1-6 acyl group, such as a formyl group and an acetyl group); or a complex protein, such as a so-called glycoprotein formed by the binding of sugar chains to a polypeptide, or the like.

A partial polypeptide of the polypeptide of the present invention may be any partial peptide of the above-mentioned polypeptide of the present invention and has activity which has substantially the same characteristics. For example, a polypeptide that is used herein comprises a sequence of at least 10 or more, preferably 50 or more, further preferably 70 or more, farther more preferably 100 or more, and most preferably 200 or more amino acids of the amino acid sequence comprising the polypeptide of the present invention, and, for example, has biological activity substantially the same characteristic with the function of the polypeptide of the present invention. An example of a preferable partial polypeptide of the present invention contains each functional domain. Further, the partial peptide of the present invention normally has a carboxyl group (—COOH) or a carboxylate (—COO—) at the C-terminus, and it may also have an amide (—CONH₂—) or an ester (—COOR) at the C-terminus like the above polypeptide of the present invention may have. Further, examples of the partial peptide of the present invention, similar to the polypeptide of the present invention described above, include a peptide wherein an amino group of a methionine residue at the N-terminus is protected with a protecting group; a peptide wherein a glutamyl residue at the N-terminus which is generated by in vivo cleavage is pyroglutamated; a peptide wherein a substitution on the side chain of intramolecular amino acids is protected with an appropriate protecting group; a complex peptide, such as a so-called glycopeptide formed by the binding of sugar chain to a peptide, or the like. The partial peptide of the present invention can be used as, for example, a reagent, reference materials for experiments, or an immunogen or a portion thereof.

Particularly preferred salts of the polypeptide of the present invention or the partial peptide are physiologically acceptable acid-added salts. Examples of such salts that are used herein include a salt formed with inorganic acid (for example, hydrochloric acid, phosphoric acid, hydrobromic acid and sulfuric acid), and a salt formed with organic acid (for example, acetic acid, formic acid, propionic acid, fumaric acid, maleic acid, succinic acid, tartaric acid, citric acid, oxalic acid, benzoic acid, methane sulfonic acid and benzenesulfonic acid).

The polypeptide of the present invention, the partial peptide thereof or salts thereof or amides thereof can be prepared by a chemical synthesis method known in the technical field.

For example, amino acids whose α-amino groups and side chain functional groups are appropriately protected are condensed on resin (which is commercially available resin for protein synthesis) in accordance with the sequence of a target polypeptide, according to various condensation methods known in the art. Various protecting groups are then removed simultaneously with cleavage of the polypeptide from the resin at the end of reaction. Further, reaction for forming an intramolecular disulfide linkage is conducted in a highly diluted solution, thereby obtaining a target polypeptide, the partial peptide thereof or amides thereof Examples of activation reagents that can be used to condense the above protected amino acids include those that can be used for polypeptide synthesis and are represented by carbodiimides, such as DCC, N,N′-diisopropylcarbodiimide and N-ethyl-N-(3-dimethylaminopropyl) carbodiimide. For activation by such reagents, both protected amino acids and a racemization-suppressing additive (for example, HOBt or HOOBt) are directly added to the resin; or protected amino acids can be previously activated with acid anhydride as a control, or HOBt ester or HOOBt ester, and then added to the resin.

Solvents used for activation of protected amino acids and condensation with resin can be appropriately selected from solvents known in the art as applicable to polypeptide condensation reaction, such as acid amides, halogenated hydrocarbons, alcohols, sulfoxides and ethers. A reaction temperature is appropriately selected from a known range that can be used for reaction of polypeptide linkage formation. Activated amino acid derivatives are normally used in 1.5 to 4-fold excess. When condensation is insufficient as a result of a test using ninhydrin reaction, condensation reaction without eliminating protecting groups is repeated for sufficient condensation. When condensation is still insufficient, unreacted amino acids are acetylated using acetic anhydride or acetylimidazole so as not to affect the subsequent reaction.

Protecting groups being normally employed in the technical field can be used for raw materials, such as those for each of amino groups, carboxyl groups and serine hydroxyl groups.

The protection of functional groups that should not involve the reaction of raw materials, protecting groups, and the elimination of the protecting groups, and the activation of functional groups that involve reaction and the like can be appropriately selected from known groups or performed by known measures.

The partial peptide of the present invention or a salt thereof can be produced according to a peptide synthesis method known in the technical field, or by cleaving the polypeptide or the present invention with appropriate peptidase. For example, the peptide synthesis method may be either a solid-phase synthesis method or a liquid phase synthesis method. Example of a known condensation method and a method of elimination of protecting groups are described in Nobuo IZUMIYA et al., Basics and Experiment for Peptide Synthesis, Maruzen (1975); Haruaki YAJIMA and Shunpei SAKAKIBARA, Experiment Course for Biochemistry 1, Protein Chemistry IV, 205 (1977); and Development of Pharmaceutical Preparation, vol. 14, Peptide Synthesis, under the editorship of Haruaki YAJIMA, Hirokawa Publishing Co.

After reaction, the partial peptide of the present invention can be purified and isolated using known methods, such as solvent extraction, distillation, column chromatography, liquid chromatography, re-crystallization and the like in combination. When the partial peptide obtained by the above methods is an educt, it can be converted to an appropriate salt by a known method. Conversely, when the peptide is obtained as a salt, it can be converted to an educt by a known method.

The antibody for the polypeptide of the present invention, the partial peptide thereof or salts thereof may be either a polyclonal or a monoclonal antibody, so far as it can recognize these substances. The antibody for the polypeptide of the present invention, the partial peptide thereof or salts thereof can be produced using as an antigen the polypeptide of the present invention or the partial peptide thereof according to a known method for producing antibodies or anti-serum.

The antibody of the present invention can be used to detect the polypeptide of the present invention and the like which are present in a specimen, such as body fluid, tissues or the like. In addition, the antibody can be used for preparing an antibody column to be used for purifying these substances; detecting the polypeptide of the present invention in each fraction upon purification; analyzing the behavior of the polypeptide of the present invention within the cells of a specimen; and the like.

The use of the DNA, the polypeptide and the antibody of the present invention will be further described below.

Using as a probe the DNA of the present invention, the antisense DNA of the DNA of the present invention, or a gene construct containing these DNAs, abnormalities (of the gene) in DNA or mRNA encoding the polypeptide of the present invention or the partial peptide thereof can be detected.

The DNA, the antisense DNA or the gene construct of the present invention are useful as a genetic diagnostic agent for, for example, damages, mutation or hypoexpression in the DNA or mRNA, and an increase or hyperexpression of the DNA or mRNA. The above gene diagnosis using the DNA of the present invention can be performed by, for example, a known northern hybridization or a PCR-SSCP method (Genomics, 5:874-879 (1989), Proc. Natl. Acad. Sci. USA, 86:2766-2770(1989)).

Moreover, for patients who cannot exert normal in vivo function because of abnormalities or deletion in the DNA or the gene of the present invention, or because the expression amount of the DNA or the gene of the present invention is reduced, it is effective that the DNA or the gene construct of the present invention is introduced for expression into the bodies of the patients by gene therapy using as vehicles appropriate vectors, such as retrovirus vectors, adenovirus vectors and adenovirus-associated virus vectors according to known techniques. Further, when patients cannot exert normal functions because of an increased expression amount, introduction of antisense can be effective.

The DNA, the antisense DNA of the present invention, or the gene construct thereof can be administered alone, or in combination with an adjuvant to promote uptake using a gene gun or a catheter, such as a hydrogel catheter.

In another example, injection of the polypeptide of the present invention or the like into patients with the above diseases also enables the polypeptide of the present invention or the like to exert its function in the patients.

Furthermore, the antibody of the present invention can be used for quantitatively determining the polypeptide of the present invention in a test liquid by a known method. Specifically, the antibody of the present invention can be used for quantitative determination by a sandwich immunoassay using monoclonal antibodies, detection by tissue staining, and the like, by which, for example, diseases that involve the polypeptide of the present invention or the like can be diagnosed.

For these purposes, an antibody molecule itself can be used, or the antibody molecules F(ab′)2, Fab′ or Fab fractions can be used. Quantitative determination methods for the polypeptide of the present invention using the antibody of the present invention are not specifically limited. Any measurement method can be used, so far as it involves detecting the amount of antibodies, antigens or antibody-antigen complexes corresponding to the amount of antigens (for example, protein amount) in a test liquid by chemical or physical means, and calculating with a calibration curve which has been prepared using a standardized solution containing a known amount of antigens. For example, nephrometry, competitive assay, immunometric assay and sandwich assay are preferably used, and a later described sandwich assay is preferred in terms of sensitivity and specificity. Examples of a labeling agent that can be used in a measurement method using a labeling substance include a substance known in the technical field, such as radioisotopes, enzymes, fluorescent materials and light-emitting materials.

Details about the general technical procedures concerning these measurement and detection methods can be referred to in a review, reference book or the like, such as Radioimmunoassay 2 edited by Hiroshi IRIE, (Kodansha, issued in 1979); Enzyme Immunoassay edited by Eiji ISHIKAWA et al., (3^{rd} edition; Igakushoin, issued in 1987); and Methods in Enzymology (issued by Academic Press), vol. 70, “Immunochemical Techniques (Part A)”, vol. 73, “Immunochemical Techniques (Part B)”, vol. 74, “Immunochemical Techniques (Part C)”, vol. 84, “Immunochemical Techniques (Part D: Selected Immunoassays)”, vol. 92, “Immunochemical Techniques (Part E: Monoclonal Antibodies and General Immunoassay Methods)”, and vol. 121, Immunochemical Techniques (Part I: Hybridoma Technology and Monoclonal Antibodies)”.

Moreover, DNA chip prepared by arraying the DNA of the present invention is useful in detecting mutations and polymorphism of the DNA of the present invention, and monitoring the DNA dynamics. Regarding DNA array, which is a type of DNA chip, see “DNA microarray and Current PCR method” (a supplementary volume of Cell Technology, Genome Science Series 1, under the editorship of Masaaki MURAMATSU and Hiroyuki NABA, 1^{st} edition, issued on Mar. 16, 2000) and the like.

Further, polypeptide chip prepared by arraying the polypeptide of the present invention can be a strong tool for functional analysis on the expression, interaction and posttranslational modification of the polypeptides of the present invention, and for identification and purification of proteins.

Antibody chip prepared by arraying antibodies against the polypeptides of the present invention is very useful in analyzing the correlation between the polypeptides of the present invention and diseases, disorders, or other physiological phenomena.

Methods and materials for preparing the chips are public-knowledge to persons skilled in the art.

Furthermore, the polypeptides of the present invention or the like are useful as reagents for screening compounds which interact specifically with these substances. More specifically, the present invention provides a method for screening compounds specifically interact with the polypeptide of the present invention, a partial peptide thereof or salts thereof by using these substances or antibodies against them; and provides the screening kit therefor.

Compounds or salts thereof that are identified by using the screening method or the screening kit of the present invention interact with the polypeptide of the present invention or the like. For example, the compounds regulate, inhibit, promote or antagonize the biological activity of the polypeptide of the present invention or the like. The compound or the salt thereof may directly act on the activity of the polypeptide of the present invention or the like, or indirectly act on the activity of the polypeptide of the present invention or the like by acting on the expression of the polypeptide of the present invention or the like. An example of the salt of the compound that is used herein is a pharmaceutically acceptable salt. Specific examples of such salts include a salt formed with inorganic base, a salt formed with organic base, a salt formed with inorganic acid, a salt formed with organic acid, and a salt formed with basic or acidic amino acid. Compounds that inhibit the biological activity of the polypeptide of the present invention or the like can also be used as pharmaceutical preparations, such as therapeutic agents and preventive agents for each of the above-mentioned diseases.

When nucleotides (bases) and amino acids are indicated with abbreviations in the present specification, the abbreviation follow the IUPAC-IUB Joint Commission on Biochemical Nomenclature, or those commonly used in the art. Amino acids for which optical isomerism is possible are, unless otherwise specified, in the L form.

### BEST MODE FOR CARRYING OUT THE INVENTION

The present invention will now be further described by means of examples that are not intended to limit the present invention. The various gene manipulations employed in the examples are according to the methods described in the above Current Protocols in Molecular Biology (edited by Frederick M. Ausubel et al., 1987).

(1) Construction of cDNA Library Derived from Human Adult Whole Brain and Human Embryonic Whole Brain

Double-stranded cDNA was synthesized using an oligonucleotide having Not-I site (GACTAGTTCTAGATCGCGAGCGGCCGCCC(T)₁₅) (Invitrogen) as a primer, mRNAs (Clontech) derived from the human adult whole brain and human embryonic whole brain as templates, and SuperScriptII reverse transcriptase kit (Invitrogen). Next, an adaptor (Invitrogen) having SalI site was ligated to the cDNA, followed by digestion with NotI and 1% low-melt agarose electrophoresis. Thus, DNA fragments of 3 kb or more were purified.

The purified cDNA fragment was ligated to pBluescript IISK+ plasmid pre-treated with SalI-NotI restriction enzymes. The recombinant plasmid was introduced into Escherichia coli strain ElectroMax DH10B (Invitrogen) by electroporation.

(2) Screening

The terminal nucleotide sequences of clones were determined. Using the obtained sequences as queries, a homology search program BLASTN 2.2.1 (Stephen F. Altschul, Thomas L. Madden, Alejandro A. Schaffer, Jinghui Zhang, Zheng Zhang, Webb Miller and David J. Lipman (1997), “Gapped BLAST and PSI-BLAST: a new generation of protein database search programs”, Nucleic Acids Res. 25:3389-3402) was run on nr database (GenBank+EMBL+DDBJ+PDB sequences which do not contain EST, STS, GSS or HTGS (phase 0, 1 or 2) sequences). As a result, the 5′ and 3′ terminus sequences of novel genes, for which no homologous gene was present, were related to human genome sequences (ftp://ncbi.nlm.nih.gov/genomes/H sapiens/) using a homology search program BLASTN 2.2.1.

Next, encoded genes were extracted from the genome regions interleaved in these termini by using Genscan program (Burge, C. and Karlin, S. 1997, “Prediction of complete gene structures in human genomic DNA”, J. Mol. Biol. 268, 78-94). Using the obtained genes as queries, a homology search program BLASTN 2.2.1 was run on mergedb (Kazusa DNA Institute), and then cDNA full-length analysis was performed for the confirmed novel long chain genes (Genscan prediction cdp is 1200 bp or more).

For sequencing, a DNA sequencer (ABI PRISM377) and a reaction kit, which are manufactured by PE Applied Biosystems, were used. Most sequences were determined by a dye terminator method using shotgun clones. Part of the nucleotide sequences was determined by synthesizing oligonucleotides based on the determined nucleotide sequences, then performing a primer walking method.

As described above, screening for novel DNAs or genes was performed. As a result, a novel DNA or gene represented by any one of SEQ ID NOS: 1 to 3 in the sequence listing was detected.

The nucleotide sequences of these novel DNAs or genes were determined by the above sequencing method. Table 1 shows the names of clones having the DNA or the gene of the present invention, the length of a polypeptide encoded by the gene in the clone, its putative function.

TABLE 1
Clone Name and Putative Function

| SEQ | Clone | | | |
|---|---|---|---|---|
| ID | | Protein | Full length or | |
| NO: | Name | length | partial sequence | Putative function |
| 1 | ff00654 | 591 | Partial sequence | Motor biomolecule. |
| | | | | Involved in |
| | | | | chromosome movement |
| | | | | in spindle |
| | | | | mitosis by arranging |
| | | | | nervous intracellular |
| | | | | transport and Golgi |
| | | | | body position. |
| 2 | fh11694 | 227 | — | |
| 3 | pj01991 | 917 | — | |

(3) Homology Search for the DNA of the Present Invention

Next, based on the thus obtained nucleotide overall sequences, the amino acid sequences of the clones were searched on the library of known sequences, nr, using an analysis program BLASTP 2.2.1 (the above-mentioned “Gapped BLAST and PSI-BLAST: a new generation of protein database search programs”). Thus, it was shown that the clones were homologous to each homologous genes listed in Table 2. Table 2 shows the information on these homologous genes, specifically, name, database ID, biological species, nomenclature, protein length and the literature containing the information.

TABLE 2
Homologous Gene of Each Gene and Biological Species

| | Homologous gene | | | | |
|---|---|---|---|---|---|
| SEQ | | | Bio- | | |
| ID | | | logical | Protein | |
| NO: | Name | Database ID | species* | length | Literature |
| 1 | dynein heavy | pir\|\|T30878 | Tg | 1136 | Mol. Biol. Cell |
| | chain | | | | 5(1), |
| | isotype 4 | | | | 57-70 (1994) |
| 2 | KIAA1661 | gi\|14779561 | Hs | 154 | — |
| | protein | | | | |
| 3 | hypothetical | gi\|12232415 | Hs | 875 | — |
| | protein | | | | |
| | FLJ21610 | | | | |

*In Table 2, nomenclature and meaning of each biological species brevity code is as follows:
Hs: human = Homo sapiens;
Tg: Hawaii uni or echinoid = Tripneustes gratilla

Table 3 summarizes a variety of data concerning homology between the DNA or the genes of the present invention contained in each clone and each homologous gene listed in Table 2. The meaning of each item in Table 3 is as follows:

Score: the higher the value, the higher the reliability

E-value: the closer this value to 0, the higher the reliability

Homology: the identity proportion (degree) of amino acid residues in a homologous region

Homologous region %: the proportion (%) of a homologous region in a homologous gene

TABLE 3
Homology between each gene and homologous gene

| | Homologous region | | | | Homology value | | | |
|---|---|---|---|---|---|---|---|---|
| SEQ | | | Homol- | | | | | Homol- |
| ID | | | ogous | | | E- | | ogous |
| NO: | Clone | | gene | | Score | value | Homology | region % |
| 1 | 50 | 550 | 237 | 737 | 802 | 0 | 76%(385/501) | 44% |
| 2 | 148 | 227 | 35 | 114 | 110 | 1e−23 | 71%(57/80) | 52% |
| 3 | 47 | 917 | 7 | 875 | 638 | 0 | 43%(416/958) | 99% |

(4) Search for Each Domain

Using as queries the amino acid sequence encoded by DNAs contained in the clones, functional domains were searched with a search tool contained in Pfam 6.6 (Pfam HMM ver. 2.1 Search (HMMPFAM), Sonnhammer, E. L. L., Eddy, S. R., Birney, E., Bateman, A., and Durbin, R. (1998) Pfam: multiple sequence alignments and HMM-profiles of protein domains“, Nucleic Acids Res. 26:320-322).

Further, transmembrane domains were searched with a prediction program for membrane proteins, the SOSUI system (ver. 1.0/10, March 1996) (Takatsugu Hirokawa, Seah Boon-Chieng and Shigeki Mitaku, SOSUI: Classification and Secondary Structure Prediction System for Membrane Proteins), Bioinformatics (formerly CABIOS) 1998 May; 14(4): 378-379).

Table 4 shows the detected functional domains and transmembrane domains for each clone.

The meaning of each item in Table 4 is as follows:

Functional domain: a domain detected by Pfam or SOSUI

Starting point (From): an amino acid position as a starting point of a functional domain

End point (To): an amino acid position as an end point of a functional domain

Score (Pfm only): the higher the value, the higher the reliability

Exp (Pfam only): the closer the value to 0, the higher the reliability

The complete notation of “SAM” in the functional domain column is “SAM domain (Sterile alpha motif)”.

TABLE 4
Functional domain

| SEQ | Clone | | | | | Homologous gene | | | | |
|---|---|---|---|---|---|---|---|---|---|---|
| ID | Functional | | | | | Functional | | | | |
| No: | domain | From | To | Score | Exp | domain | From | To | Score | Exp |
| 1 | | — | — | — | — | — | — | — | — | — |
| 2 | sosui | 139 | 161 | — | — | — | — | — | — | — |
| 3 | SAM | 850 | 914 | 10.6 | 0.31 | SAM | 808 | 872 | 9.4 | 0.43 |

(5) Expression Site

Expressions in the tissues and the sites of the brain were examined by RT-PCR ELISA (Nagase, T., Ishikawa, K., Suyama, M., Kikuno, R., Miyajima, N., Tanaka, A., Kotani, H., Nomura, N. and Ohara, O. Prediction of the coding sequences of unidentified human genes. XI. The complete sequences of 100 new cDNA clones from brain which code for large proteins in vitro. DNA Res. 1998 Oct. 30; 5(5): 277-86). Table 5 shows the result.

The expression amount (unit (fg) per ng of poly(A)+ RNA) is represented by + for less 0.1; ++ for more than 0.1, less than 100; and +++ for more than 100. In addition, shows those are not investigated.

Table 6 shows the complete notation of each tissue and site of the brain.

TABLE 5
Expression site of each gene

| SEQ | Adult | | | | | | | | | | | | | | | | | | | Embryo | |
|---|---|---|---|---|---|---|---|---|---|---|---|---|---|---|---|---|---|---|---|---|---|
| ID | Tissue | | | | | | | | | | Site of the brain | | | | | | | | | Tissue | |
| NO: | He | Br | Lu | Li | Sm | Ki | Pa | Sp | Te | Ov | Am | Co | Ce | Ca | Hi | Ni | Nu | Th | Sp | Li | Br |
| 1 | + | + | ++ | + | + | + | + | + | ++ | ++ | + | + | ++ | ++ | ++ | + | + | + | ++ | + | + |
| 2 | − | − | − | − | − | − | − | − | − | − | − | − | − | − | − | − | − | − | − | − | − |
| 3 | − | − | − | − | − | − | − | − | − | − | − | − | − | − | − | − | − | − | − | − | − |

TABLE 6
Complete notation of each tissue and site of the brain

| | Abbreviated | |
|---|---|---|
| | notation | Complete notation |
| Tissue | Br | Brain |
| | He | Heart |
| | Ki | Kidney |
| | Li | Liver |
| | Lu | Lung |
| | Ov | Ovary |
| | Pa | Pancreas |
| | Sm | Skeletal muscle |
| | Sp | Spleen |
| | Te | Testis |
| Site of | Am | Amygdala |
| the brain | Ca | Caudate nucleus |
| | Ce | Cerebellum |
| | Co | Corpus callosum |
| | Hi | Hippocampus |
| | Ni | Substantia nigra |
| | Nu | Subthalamic nucleus |
| | Th | Thalamus |
| | Sp | Spinal cord |

(6) Chromosome Position

Using the DNA nucleotide sequences of the clones as queries, an analysis program BLASTN 2.2.1 (the above-mentioned “Gapped BLAST and PSI-BLAST: a new generation of protein database search programs”) was run on Genbank release 122 and 123, human genome sequences corresponding to the library of known sequences. Additionally, using the DNA sequences of the clones as queries, the homology search program BLASTN 2.2.1 was run on clone libraries (ftp://ncbi.nlm.nih.gov/genomes/H sapiens/) encoding human genome.

The description of the chromosome number from which the clone had been derives was extracted from the definitions for the matched clones as listed in Table 7.

TABLE 7
Chromosome position of homologous gene

| | Chromosome |
|---|---|
| SEQ ID NO: | position |
| 1 | 12 |
| 2 | 17 |
| 3 | 2 |

According to the above information on homology, homologous genes, domains, expression sites, chromosome positions and the like, a person skilled in the art can predict that the DNAs or the genes of the present invention respectively have functions described in Table 1.

Industrial Applicability

A single nucleotide polymorphism, SNP, which is a change in one base (nucleotide) among individuals in the DNA or the gene of the present invention, can be found by performing PCR using synthetic DNA primers prepared based on the nucleotide sequence of the DNA or the gene of the present invention or a part thereof, and using chromosome DNA extracted from human blood or tissue so as to determine the nucleotide sequence of the product. Therefore, individual constitution or the like can be predicted, which enables the development of a pharmaceutical preparation suitable for each individual.

Further, when ortholog (homolog, counterpart) genes for the DNA or the gene of the present invention in model organisms, such as mice, are isolated with cross hybridization, for example, these genes are knocked out to produce human disease model animals, so that the causative genes which cause human diseases can be searched and identified.

Novel DNAs or genes obtained by the present invention are assembled on a so-called DNA chip, and then probes prepared using blood or tissue derived from cancer patients or patients with diseases that relate to the brain, such as mental disease, or as a control using blood or tissue from healthy individuals are hybridized to the chip, so that the chip can be applied to diagnosis and treatment for the diseases.

Moreover, antibody chip, on which the antibodies against the polypeptides of the present invention are thoroughly prepared and arrayed, can be applied to diagnosis, treatment of diseases and the like through proteome analysis, such as detection of a difference in expression amount of a protein between a patient and a healthy individual.

Furthermore, the DNAs or the gene constructs of the present invention can be used as an active ingredient of vaccine.

The present application asserts priority based on the specification of Japanese Patent Application No. 2002-205915 and includes by reference all of the contents as disclosed in the specification.

1. DNA comprising a nucleotide sequence encoding a polypeptide (a) or (b) as follows:
(a) a polypeptide comprising an amino acid sequence which is identical or substantially identical to an amino acid sequence represented by any one of SEQ ID NOS: 1 to 3;
(b) a polypeptide which comprises an amino acid sequence derived from an amino acid sequence represented by any one of SEQ ID NOS: 1 to 3 by deletion, substitution or addition of a section of amino acid(s), and has biological activity which is substantially the same characteristic with the function of the polypeptide of (a).

2. DNA hybridizing to the DNA of claim 1 under stringent condition, and encoding a polypeptide having biological activity which is substantially the same characteristic with the function of the polypeptide of (a) of claim 1.

3. A gene construct containing the DNA of claim 1 or 2.

4. A polypeptide (a) or (b) as follows:
(a) a polypeptide comprising an amino acid sequence which is identical or substantially identical to an amino acid sequence represented by any one of SEQ ID NOS: 1 to 3;
(b) a polypeptide comprising an amino acid sequence derived from an amino acid sequence represented by any one of SEQ ID NOS: 1 to 3 by deletion, substitution or addition of a section of amino acids, and having biological activity which is substantially the same characteristic with the function of the polypeptide of (a).

5. A recombinant polypeptide, which is encoded by the gene construct of claim 3.

6. An antibody against the polypeptide of claim 4 or 5.

7. A DNA chip, on which the DNAs of claim 1 or 2 are arrayed.

8. A polypeptide chip, on which the polypeptides of claim 4 or 5 are arrayed.

9. An antibody chip, on which the antibodies of claim 6 are arrayed.


SEQUENCE LISTING (7 sequences)

SEQ ID NO: 1 - DNA, 10591 bp, Homo sapiens
  CDS (4005)..(5777)
ctggtcttgaactcttgacctcgggtgatccacccaccttggcgtcccaaagtgctgggattacaggcatgaacaactatgcttggcccagttacagccttaataataggtttaggtcaaaataaagaagcaatgtatttctggagtgtgattcctacagatggttctgttctttaaattaagatagtgaagattttggtaacacagatgtttcttcaaattagttcccaatttgccatatttaaaaatgaaggacccaccctggtatttctggaagttagagatggaatttgcttatttgtagagcagctgcactggaacacctgcccccgttttcttgcatttgtagaatgatgctccattgcttttgaatcattttcttttttctttttttttgtattattatcttctatcaaaaagactctggcgaaaaagatgacggttctgtataagctggcccgggagcagctgtccaagcagtatcactatgattttggactcagagccctgaaatcggtgctggtcatggctggtgagctgaagagaggctcctctgaccttagggaggtaggggccacgtgctggaacattctctggtttcagctgcttcaggcatttactacgtgccattggggaggtgatgggcacagtatggtatcacctgaaaggtttaagtcttagctgcctcttcaccccgcggtctggccttactttggcctcttacctgttgtatcgtttgcttgtaattacacttagtaccttaaggtcaagtgcaatgtcttcgctccaatagcaacatttatgatgcttccatagtaataatctgtagtctcaggatgggtgtggcggaagggtggggactaacaaaattctcatgaaagcatgaaggtgagttgttattttctctttgaaacaaaagaggggaacaattgtgttttttttacatggactttaaataaaaggtttagtcaatgtttctgtgggcaacaggcttacctgtttctctagtgactgtactcaatgttgcatgcacacacgcacgcacacacacatttgggtagtggggggaaaacagccgtgagtgcagtgatggaagcctgtttgtgtccataggaggagaggctgccacaaatcgcttcattcctttgggattttcacacatttgggttttttttttttttttttttggaggggtcgaaggaagagttatttatgctcttacaaaaaaggaagctgtaaaatgtaaaacactataaagtcagatggagctgcattcgtcatttgtacttcttttttcttaggagaaaggagttatatggaaacttctttcattttctcccctgctcctctatgtccaaagttgaagggcgatcatgctattgagtgggtcatatcttctgaactgaaaaaccaccgatgccactttcagtcttagcaacagggaaaacagtctcaagtgctatctaaggctctacgaagaagagtggggccgtttgcagcagtctctcctcctagggaggtgaatatggtcccatcactgcactcactactggcttgttttcttttactgccttacactgaacccaagcatgcattcataggctcaactgctgcacaatctggtacttccttagaagaatgaaaacacattgcaaataccgaatatccaaggacttccgagaaagaggtaaaaaacagcctccttttctaggaggaaaatccctcttctcatccagcaatgtggacttacacatgtaaaactgtgtttgtacagagactgcgatggtttcttcacataggaaattaactttgctgtattgtctttgggcttctttatcacattaaaagtttgcaagcttggacctttgttcttattcagtaaatctatgtcacaggatgagtgaggatttagcatcttttgcttcaaattagaaccacatatttggaatttactgctgcagttctcagctgcgcttatcatgacagtggtggaaactggaggacattaaaaagcccatgaagtcagctgtacttgccatttatttagtcttgtttggaataaaaggtaaagaattcacaaataccaccacctcagcacctgtttcaaaggaaacatcaggcttcagcttgcaacatgatgtcgttacaattaagcaaatagcagaagcccccgtcagttgcttccttcttctggagaaggcggggaactgcaatccccggggctcctgtgttgagtctgggaggcagaacaaaaggaaaagtggccttttccactccacatcctggaggtaatgggtagagaagacatacccacatttccagatagtgaacgctgagtagagaagcaggactacctgtcctaggagacaacatagagacgcttgccaggtcctgagttcagaattttgtttttgagatttatataaaagcacctaaggctgggcgcagtggctcatgcctgtggtcctagcactttcggaggctgaggtgggaggattgcttgaggccagaagtttgagaccagcctgggcaacatggcaaaaccccatttctttccaaacaaatacccagaactaaaaaatttagctggctgtggtggcacacttctgtggtcccagctacttgggaggctgaggtaggaggattgcttgggcctgggaggtggaggctgcagtgagccgagatggcactattgcactccagcctgggtgacagagcgagaccctgtctcaaaaaaaaaaaaaaaaaaaataataaaaagtatatatatatatgcgcgcacctaaaggtcaaagtaaatgttcttcctgtaataaggaacctttgctttagcgtagatggccatacccctctgtaaggtggggaggaggaagtttccgctccttccctctggccgagatctgttttcttctccttgcacggcccaccaggacactcagcctcctaggagcaggcccaccctggcctcactcctctgcctgtgctcctattttatattttatttaatttttacatttatatacaattttgtattgtatatttcacttttatatattttattttacatttttattttcatatgaaactttgaaaaatgcatatttacatttatattatttatatgatcattattaaatatgaaatatgaaatttcatatattcatattacacataatatcttatacatattttatactatataatatacataacattttatatacatttataaatttatataatttataaataatacaaatataattaatttatatgaatgatataaaatatataaattttataaaatttaatttatataaaattgtaatatatgtatataatttatatataattttatatttcattttaaattttatatttcatttaaatatatatgtatgtgtatatatatgtgtgtgtgtgtgtatatctatctatctatctatctttttttcgagacagagtttcactcttgtcacgccaggagtgcgatggcgcaatctcggttcactgcagcctccacctcctgggttcaagcgattctcctgcctcagcctcccgagtagctgggattacgggcacctgccgccacacccagctaatttttgtgtttttagtagagatgtgatttcgccatgtcggccaggctgatctcgaactcctcaggttgtccacccacctcagcctcccaaggtgctggaattgcaggcatgagccactgcacccggccatatatgtgtatattatataatatgaagtacatattatatgaaatatatgaaatatatattttatattttatgtttaatctcatgtaaactattttatataatttatatttatttacatattaatataaaataaaaaataaaattatataatttctgtatataaaaatatataatacatatattcagaaattatataattttaaattaatttctgtaatttaaatttaaattaatttaaattaatttctgtaatttaaaattatgtaattttaaatgtgtataaaattgttaaaaataataaaatgtttatataaaaattatattttatatttcataagagtgttaccactgggttggaaacacagtccaaagcactgggttcctttggaaatctcttggaaacatgtgtttcatttcctgcaggacgtggtgctgatgagggccttgcgagacatgaacttgcccaaatttgtgtttgaagatgttcctcttttccttggtttgatttcggatctgtttcctgggctggactgccctcgcgtccgctaccctgacttcaacgatgcggtagagcaggtcctggaggagaacggctacgcggtcctacccatccaggtggataaagtggttcaaatgttcgagaccatgttaacccgccacacgacgatggtggtggggcccaccagagggggcaagtccgtcgtcattaacactctgtgtcaggcccagaccaagcttgggctgacgacaaagttgtacatcctgaaccccaaagccgtgagtgtcatagaactctacggcatcctggacccaaccacccgagactggacagatggggtgttgtcaaacatcttcagggaaatcaacaagccaacagacaagaaggagcgaaagtatattttatttgatggtgatgtggatgctctatgggtggaaaacatgaattctgtgatggatgacaacaggttgttgacattggccaacggggaacgcatccggctccaagcacactgtgccctgctctttgaggttggagatttacagtatgcctcccctgcaactgtctctcgatgtggaatggtttatgtggatcctaaaaacttgaaatatcgaccatactggaaaaaatgggttaatcaaataccaaacaaggtggagcaatacaatttgaatagtctctttgagaagtatgtgccctatctcatggatgtgatagtggaaggaattgtggatggaagacaagcagaaaagctgaagacaatagttcctcagacagacctcaatatggtaacccagttagccaagatgttggatgcgttgctagaaggagaaatagaagaccttgacctgctggagtgctacttcctggaggctttgtactgctctctgggagcctccctgcttgaggatggaaggatgaaatttgacgaatatatcaaacgccttgcttctttgtctactgttgacacagaaggagtttgggccaaccctggggaactgccaggtcaacttccaaccttgtatgactttcattttgataacaaacggaatcaatgggtcccatggagtaaattagttccagagtatattcatgcccccgagaggaaattcatcaacatcctggttcacacagtggataccactcggactacctggatattggaacaaatggttaaaattaagcaacctgttatttttgttggtgaatctggcacttctaagacagccactacccagaatttcctcaaaaatctgagtgaagaaactaacattgtgttaatggtcaacttctcctcccgcaccacgtccatggatatccaaagaaatttagaagcaaatgtggaaaagcgaaccaaagatacttacggcccacccatgggaaaacgcctgctggtgttcatggatgacatgaatatgccaagggctggagtgcaatggcgtgatctcggttcgctgcgacctccgcctcctgggttcgggcgattctcctgcctcggcctcctgagtggcaggaattacagacgtttcatgagagcattgtggctgtgagtggcaagctgacattctgcacgctagcactttacaaaaatattgtgcaagacctacctcccactccgtcaaagttccattacatcttcaaccttcgagatctctcacgggtttttaatggtcttgtcctcactaacccggagcgattccagacggtggcccagatggtgagagtctggaggaatgagtgtctgagagtcttccacgaccggctgatcagtgaaacagacaagcagctggtacaacagcacataggcagcttggttgtggaacattttaaagatgacgtggaggtggtgatgagggatcccatattgtttggagacttccagatggctctgcacgaaggagaaccacgcatttatgaagacatccaggactacgaggcggccaaggctctgttccaggaaattcttgaagagtataatgaaagcaacaccaaaatgaacttggttctcttcgacgatgctctggagcatttaacccgggtgcaccgtatcatccgcatggaccgcggccacgccctgctggtcggggtagggggctcagggaagcagtctctttcgaggctggctgccttcacagccagctgtgaggtgtttgagatcctgctgagccgaggctactcggagaacagtttccgggaagacctgaagagcctctatttgaaacttgggattgagaacaaagcgatgatctttctgttcacggatgcccatgtggctgaggagggcttcctggagctcatcaacaacatgctgacctcaggtacagccaaggctggcgcccgctgtggccaacaccccgctcagctcttaagggagttcactttcttcagcagttaccacctccagacactgtgggtagccctgtgcgggtgtacctgttcctccctcacggcggcccctgagataggtctcattatcttccttggctcctcctgtcctggagtctcccgagtgtgcccccttctctccgtctgcgatgatggcagtatccttgcagtaagaacaggtggtgtctgggcatctggccgtgccccgggctgtgctaagtgtgcatcatacattcccgtctccacaaaaacttgatgagaggccaggtgtggtgcctcctgcctgtgatcccagcactctgggaggccaaggagggaggatagcttgagcccaggagtttgagaccccatctttacaaattttttttttttcttttttttttttggatagagtctcgttctgtcttccaggctggagtgcagtggcgtgatcttggctcactgcaagctccgcctcctgggttcacaccattctcctgcctcagcctcccgagtagctgggactacaggcacccaccaccatgcccggctaattttttgtattttcagtagagatggggtttcaccgtgttagccaagatggtctcgatctcctgaccttgtgatccacccgcatcggcctcccaaagtgctgggattacaggcgtgagccaccgtgcctggcacaaaaatatttttaagaagttagccaggcatgatggcgtgagcctgtggttccagctactcaggaggggcaggaggattgcttgagcctgggaggttgaggctgcggtgagtcatgatcatgttatggcactccagcctgggcaacagagtgagaccctgtctccaaaaagaaaaaaaaacaaaagcaactgaaaaacaatcaaacctaatagggaaggtaccattaacatcctcatttcacagctgaggaaactgaggcccctgtagagggctgaatggcggccctctaaaagacacatcctaaacccctggatcttgagaacgagacctttgtaagaaaaagagtctttgcagatgtaattaaattaaagatcttgaagtgccttcctggatttagggtgggtcctaaatccagtgacagctgtccttataagagaaggcagagggagatttgagacacagagaataaggtcccataaagattcccacaagtcacgggacacctggagccaccagaagccagaggaggcaaagaaggatttttcccttggagctgttggcaggttaccctgagaccttgatttcaggcctccagaactgcaagagagtcggtgtctgtatttcttgtctgaagccactcgctttggggtaattagtcatggctgccccagaaatctactagagatgccacctaccttatccaagggcaccttgctgggaaggcactgaggtgagactcgaacccaggatttttagcatcttagccacgctcacaaccactttaccgtgttgttatcttgtctaccctggggccctgcaaagatccgtgctgacttcgtctggcctaatggcttacagggacctactttctttcccagggacctatttcatcgattgcttgtttgtttcagcacctgaccatattcattattgtattgctgtgttattcattgagccccagcctcccagcatttactataaaaaatttctaacataacagtgaggctgaaagaattttttctgtgaacatctgccggcatgccatctagatccctcctttctcgccttgctgcacttgctctgccacgcgtgtaaacagctttccctccctccctgcttccgcccttctcgtttttagatgcgtttcagagtcaattgcaacatcactgctctccccgctaaatgcttctgcgtgccactatctagagttcaagttttgtttagtgttttcttttgatgtaaaatttacatacagggaaacgcacaacattttaagtgtatatttgctgcattttgatcattgcgttttaagttgcgtctgtttcagtgggttgtcggctccttgagaggggggatcgggtcatccacctggatttattccacagtgtttattccagacactgggtttggtagggaataagatggtcatgacctctgccctccttggagcatacatttcagcgtgtgagtggactggttaacgacaacaacaactacacaaagagatacagcaattgcagattgtggtaagtgccgtgagtgcaaaggggagttgctgtgagagagagtgtgggcaggagcatctcagggcagacagggaaggcgtcactgtagagacttttcagctgaggcctgagagatgctgtgtgatcacactccctatggctgctgtagcaaatttccccaaacagcacggatttattcccctatgcgcctggaggtcagaaatcagaaaagggtcttaaggggttaagatcagggtgttggtggggctggttccttccagagcttcctggggacactctgtttccctgtctcttccaactgcttcaggccacccgtttcttggctctttagctcgctggctctcatccttccagtctctacttctgtcatcctgatgcaaactcagctgcttctctcttgtggggacccctgtgatcgtctccagcccacctgggtaattgagagcacgcgctccatcccaccatccttggctgcgtcacatccacagagtcctttttgcaggaaacataaagaaacccattggccggttcctgggcttaggatgccatggacacctttagagggctgttgtgcagggatcaggacgaggtggagggggtctatggcgtggaaccttgtaggtcatttataggagataagattttattctcagtgcaatgacgtagtccagatttatgggttttttaattttttagatttaattaattaattatttcagagacagggtcttactctgttgcccaggccagaatgcagcggcatgatcatagctcattgtagcttccaactcctgggcttacacaatcttcctgtcccagcctttcgagtagctgggactacagatgcatctcaccacaaccagctgattaaaaaaaaaaaaacacactttttttttaagaggtggagtcttcctgcattgcccaggctggtcttgaactcctgggctctagtgtttttcctgcctcaccttcccaaggtgttgagattacaggcgtgagccactgcacccagccaagatttatgtttctaaaagatccctccagtgcgccgtggggacgagattagaggggaacaggagtgggagaggggcgtccagtttggagctacccctgcagtgtaggcaggaagtgacagcgattggaggagggtagggcactggggacagagagaagtgggtggattcacgaggtattcaggaggcagagccaacgggacttcgatcgatttggctgtgggttgggggaggggtatcaagggtgactcccaggtttctggtggagcgagtgatcagtgcttccatttgctgagatgaggaagaccagttctgggcttgtggcattaggagcatgttgagtttgctgcactagatgagaatctgaatgtgatgtcatatagtgtattagtttgctggggctgccgtcacaaaatgccacaaactgggtggcttaaaacaatagaaatctactctcacagttctggaggccaggagcccaagatcacggtgtcggcagagcccgttccttctgagctctgggtgggggaatctgtgttgctggtggctgctgacagtctttggtgttccttggcttgtaggcacagaactccagtccctgtcttcgttgtcacgtggccttccccttgtgtgctgtctgtctctgtgtccaaatttccctcctttataagggtacctgtcatattgcattagggcccatctcaatgacctcatttcaacttgattatctctgtaaagacaatttccaaataaagtcacaatctgagctac

SEQ ID NO: 2 - protein, 591 aa, Homo sapiens [sequence omitted]

SEQ ID NO: 3 - DNA, 5438 bp, Homo sapiens
  CDS (3843)..(4523)
aaaaaaaaaaagtcatcttgtttaggcttgtctgcagtcgtgagaaaacaggagacaaaggttcaccacgtatgaagttttggttgtctgggttgaatggagtccctaaaaagatttattgaaatcacaccctctggaagctgtgactgtgaccttagttggaaatactttggtctttgcagatgtaatcaggttaggaggaggtccgtagggtgggctctaatccaataggaccatgaaagggtctcagagacctgtgggggcccatgggccccactgtgaaaactgctgctctgagccatgcctgtgctcctgccaactcctgcctgtgaccatgtaggtgtggcccacgaggagactccagcagtgagatgtgagggcgtctgctgggggttcgcaaacaggaacgcagactccaagtctcctctcagcgtctgggcactgtgggcccacccacgagcctcctgtcctgtgatacagagcagctccctggtgccacctccagatggattttctgttgctggaagttaaaccagcctcagtaattcacataatatctatttcagcattttcccacggagggggcacagggagggggcatttttcttttttcttcttccaaatttagcttgaaggctaacatgtttttcatttttaaaatagttccgtctgtgatgtgcaaacagtggaaatgcaacttgaaccagcctaagcacgatgtcgattttatggaaggatggttgggagtgtctcctgtgagctgggaagggctgaccggctgtgctgtgggaagggccaggtgtggccggcctggaagtagctgagccagggacccagtgccggcgaggccggcccacctgccgcctttgttcctcccacggtgcccgctggtcttcctgtcgctgcagaggggctccctcctcacaggagaaaacatggcccccaggtttcaccccttggagagagaacggcctcactcactctcaggtctgatccccaaattcccatataagggaattccccagtggggatcagtgcccacctccgggtgaatcaactacattgggggtctcatcatgcttggggccccgcatctctgcatcttagtgcgtcctctgagcgcacggaactgaaaccccagcagatgacgcagcttccagccgggagggccagcgggggcagaggcaacaaggtcacagctggatgaggctggtgggcgaggcaaaggccgtcgccaggcccctctctgctctcccctgcactacgggacccctgcaccgctgcccctcacacagtcccctcttcccttcagaagcagagccccccgccagtggttcccacgctggagcagggcacagcggccacacccagaggctcccattcagcactgggggcccaggaatctgcatttgcaacgtggttacagatggtggtgatgctacagctccaggaaccccactttgagaactaagtgggaggggatctctccaaacatggctgcaccagcgcccacccccgacgggagacagcacgtgccttccgagtgctcataggggaagcctgcgtccgccttccagccctgtggtctctggtcttcccagcgggtgagggccccgggcactggctctgccctttgggcacagcagggagggttcggggatggctgctggccgtggctgggacataagagacccaggatgagtgcttggggcgcttctccatccacccttgagacccctcaggccccaccttattgtctcacctgactccccccaaaccccacctcaaacctgacccccacctgaccctcacctcaaacctgacccccgctcaacccccacctctcaccatacctctcacccaacccccacccaactcccacctctcactcgacccccacctctcacccaacccccactcgacccccacccaaccctcacctgacctcttcacacccccacctcaaacctgacccccacctctcactctatcctcacccctcacctgaccccacccgacctgcacctccaacctgactctcacccaatcctccactcttatccgacccccacctgacccctacccaaccctcacctgacctcttcacacccccacctctactgcagtccggcggagcccacgtgccctactcacactggctgcgcctcctggcaccgctgagctcatggccctgttgtgttctgctcctgccccatgtgagggcacagcccacgtctgctcttctcagctgttttgctgaggacaccagacgatgtgtttctggaatactccatgccactccagcacctgaaaaaaccaggcccccgttttgcacgtgcgtggcccttctcactgggtcagcgcccctgggtccttcaggactccacccgggggtcgacagctccggcaggaagcccctctgccctggtcccatcagaaccatcagtggacacacctgtcgcctccagggcggggctgaccacagggcaatgctgccacacctccccggggagtgaactttgacttctcctccacactgactgtgcagccctggaaggtttgtagtgaacttgtagaaaacggaaaacgtgaccctgcctggcccagcggttttggtcctgtggacccagcagcctggcgtctgacttagcaattctgtgtcactgctgcttccaccggctgcagaatcggggctttcaggaacgtctccagccagctcctgagtctttccgggccccccactcatgacccgggtgaacctaacgtgcgttcacctcccatttatgtgagggtgacgttctaacaacttgagaccacgaccatgcccaggggagtgtgggctctctgcaggcatccggcgcgtttccagacacagctgaggccggacgtgctgggtcagtgagggctgagatgtgcagcctcgggcctttcctgggacactcccccgaccctgcctgtcatcccgacatgcccctcagcattcctacactcataggaatgagtcctgcactcataggaaatggggggctcaggcaggtgggtggtctgtcctcaggacaacccaaatccccgacccaaacctcttcactcccgtctcgtgctcaccaccgtgcagcctcttaatgaaccacctcccacccctcggagccaggcagggcgcgctgtgcatggggatgggtctgagctggtcttgagtcggtggaaccctggaagaagtggctccgtcattctcgttgccatgggccatctggccctgcaggtgtggatcggggacagtgaggatgcaggactggtctgcagctgcagagtccaaagacgggagcccaaagtccccatcttcaaagactatcactttgcaggtagagttccagtggattaatctgggggcacacaaacatttggtccataaaagactatcactttgcaggtacagttccagtagattaatctgggggcacacaaacatttggtccacaatagggcccatggtgggggggggacctgggcaccaccgagggtggaagcaagttgtggaatgtggagcccaccccattagagcctgggcagggaggtgggtgcggctgtccctcggcctctggtccaggggcccttggatggccctcagcactaagtgagggtgtctccaacccagcccgtggatgtgtggggccgagggttcttggttggggtgcacctgagcactgtggggtgttgggcagtgtcctggcccccgtacgttagatgccagcaacactgccacctcctacaacaatcagaaatgctcccagaattgtcagagtggggggtgctgcccttcccaacagttgaagagcactggttgacagaaatttgatttccatgtggcagcctctctcccgagagcctcatcctgtccccacactgcaaggcgtgttcccaaggttgggctccgtcctggtggctcccgccggcatctcctgggcagctgtgactgctgtgtccaccgggccggggtgtctgccgtagcccagcatgcagtggagaggatggccacggggacagcggggctgggggcaagggaggatccagaagcatccggcctggggccgacgggtagtgaccatctcagggcatccgaggttcacccacgttcgcctcccagaaaccctgagaggtggaggttatcattcctgctgctgcctgaggaaacagggactcagagaaaccgtgctggtcactgtcctgcctcggcctccctcgctggagctggtcagcactgcagcgcagccccagccttgtttggtttttattttattttatttattttgagatggagtctcgctctgtctcccaggctggagtgcaatggcgcgatctcggcccaccgcagcctctgcctcccgggttcgggtgattctcctgcctcggcctcccgagtagctgggattacaggaacgtgctaccaggccgggctaatttttgtgtttttggtagagacggggtttcgtcatcttggccaggctgatctcggactcctgacatcgtgatccacctgcctcggcctcccaaagtgctgggatggcaggcgtgaaccactgtgctcggccctggctgaataatatcattgtatggaaatgccgcatggtgtccatccgttctttccttgatggacacttgagtggcttcgtctcttggccactgtaggttgagctgctgtgaacactgtatacgagcctctgcgtggatgtgtgtttgaatttctcctgggcatatacgtaggagtagaattgctggatcatgcggtagctctccgttcgatggtttgaggaactgccagacgtgggttgtgtttttgcagcaagaactcattctgcagtttgccctgcagtgggtgttttgccgcatcagtcggcggtctctggcctacagcatggttgtggggcttggggggccctgctgtggtaggtggaggcaaggaggaacacctgccacctctaggcaatacacaaggaatttttttacataatgtgacatttttataccatcggactattggaaagcatttctttcttaagtttcattctctgatgcctagacaaaaatagactgaggaactgtaaaaagacactgaccatatttccctgaaagccaggcacttgagcattgcaattaactcttcttttttctctcctctgctgtcaagaagtaaatagaaactaacagctgagcagtttggatgtatgttagctgatgactcacgaagcattcacagccagggggccagctgctcttctgctatagaaagaaaacctattttggccgggcttggtgattcacgcctgtcatcccagcactttgggaggccgaggcgggtggatcacgaggtcaggagttcgagaccagcctggccaagatggtgaaaccccgtgtctctactaaaaatacaaaaattatccggc

SEQ ID NO: 4 - protein, 227 aa, Homo sapiens [sequence omitted]

SEQ ID NO: 5 - DNA, 4142 bp, Homo sapiens
  CDS (2)..(2752)
cgcggcccggcggggctgccaggcggcgagcgccgcggcggccccgggaggtggcggcgggcgcgagagcctgggccgcgcgggactgaccgtcggggccccgggacggcggccccggggcgcccatgccatggagaagctggcggccgggctggccggcctgcgctggagcatgggcgccttcccgctcgacctcatcgtcagccgctgccgcctgcccacgctcgcctgccttgggccaggggagtacgccgagggcgtcagtgagcgagacatcctgctcatccactcctgccggcagtggacaacggtgacagctcataccctggaggagggccactatgtcatcgggcccaagatcgacatccccctgcagtacccagggaagttcaagctcctggaacaggcccgggatgtgcgggagccagtgaggtacttcagcagcgtggaggaggtggccagtgtcttccctgaccgcatcttcgtgatggaagccatcaccttcagcgtcaaggtggtgtcgggcgagttcagcgaggacagcgaggtgtacaacttcacgctgcatgcgggcgacgagctcactcttatgggccaggcggagatcctgtgcgccaagaccaccaaggagcgctcgcgcttcaccaccctcctgcgaaagctgggccgggccggggcgctggccggggtgggcggcggcggcccagcgagcgcgggggccgcgggaggcactggcggcgggggcgccaggccggtcaaaggcaagatgccctgcctcatctgcatgaaccaccgcaccaacgaaagcctgagcctgccctttcagtgccagggccgcttcagcactcgcagcccgctggagctgcagatgcaagagggcgagcacacggtgcgcgccatcatcgagcgcgtgaggctgccggtgaacgtgctggtgcccagccggccgccgcgcaacccctacgacctgcacccggtgcgggagggtcattgctacaagctggttagcatcatctccaagacggtggtgctggggctggcgctgcgccgcgagggcccggcgccgctgcacttcctgctgctcacggacacgccgcgcttcgcgctgccgcagggcctgctggccggggacccgcgcgtcgagcgcctggtgcgcgacagcgcctcctactgccgcgagcgcttcgaccccgacgagtactccacggccgtgcgcgaggcgccagcggagctcgccgaagactgcgccagcccgcgccgcgcgcgcctctgcctgcccgcgccgcgcgcccccgggctcgcccgcgcccccggcccgctagcgccggctcccgccggcgagggcgaccaggagtacgtgagccccgactgggcagccgcgcccgagcccgccgcgccgcccgccgagatcccctacgaggagttgtgggcgcaccaggggcccgagggcctcgtccggccgcccccagggctcgacctcatctccttcggggccgcgggaccgccgcgtcgggagccggaagcgccgccgcctccagtccctcccaaatccgaggcggtgaaggaggagtgccgcctgctcaatgcccctccagtgcctccccggggtggcaatggcagcggccggctctccagcagccccccggttccccctcgcttccccaagctgcagccggtacattcccccagctccagcctctcctactactcctctggcctccaggatggggcgggttcccgcagtggcagtggctccccatcgccggacacctactccctctattgctacccatgcacctggggagactgcaaggtgggcgagtcctctagccgcccagcccccggtcccctaccctcaaccacacagcccagccaggcctcccgggccctcacagagcctctgagcggtcgagccgcctcccttctgggggctgacacccctgttaagacctaccacagctgccctcctctattcaagccctcacatccccagaagcgctttgctccgtttggagctctcaaccctttttccgggcctgcctacccctcaggcccttcagcggccttgtcttctgggcccagaaccacctcgggtcctgtggctacctctggccctgcgtattccccaggcccagcctcgccaggccaggcctattcagctgctcccccctcctcctgcgccccctcctcctcctcttcttctgaatggcaggaaccagtcctggagcccttcgatccctttgagctggggcagggcagttctccagagcctgagctgctgcgttctcaggagcccagagcagtggggacacctgggcctggaccccgcctttcaccacttggcccctccaaggcctttgagcctgaaggtttggtgctgcaccaggtccccaccccactgtcaccagctgctctgcagggacccgaggcgggaggagcactttttctaacccaagggcgcctggaagggcctcctgccagtccccgggatggagccacaggctttggagtccgagatgcctcctcctggcagccccctgctgacctgtctgcactctccctggaggaggtctctcgcagtctgcgtttcatcgggctctcagaggatgtggtgagcttctttgcccgagaacgcatcgatggtagcatctttgtgcagctcagtgaggacatcctggcagatgacttccacctcaccaagctgcaggtcaagaagatcatgcagttcatcaaaggctggaggcccaagatctgaactgcccagctggagctgcacagctggaatgctggtatgggggccccaggtacagcactccggaggagcaggtgctgcctgcaagaaggatctatgtcgagactgaggctgctcagcagccactgggtggatccaggggagatgcatgtggaaatgtggtcctctggggtcagacccctgcacgggacatcttgcctttgagtgtgcagagtacatggggaaggggctgggggcaccactgtgtacctgggcccagtaaggcatttgccgtgattcccacaacggggtcaaaagctggccttcagggtgacctaacaccacctcatgccctgctatagaccttcacaaacgacttccactgctgaagcctgtaggctctgtttagagacaagaagatggctggtaatttaagcaccgatttcccaagtgcccactctcctttgtgctctgttggcttttggcctaaagctgccccagagtgagggtgtagatgtctgtgtctgtgagatgcctttcccttccccctctgctccaccgtggttgagggaggtggggcctggccccgcacacaggtgagtcgtgtacaagcctaacccatggcacctcagaggctccacctgtgggtcctggttcatgggtgacaactttggaagagcacagctctgcaattgacccgacccacttatctagttaggaagccagacactgcccagatgactctagcaccctggcttctgctctgactttactgcagctagtcatccatccgtcaggtggcgttcagtctcagaatactgattccgtggaagagcaggttgatttaggtcagcttctccttgattctagaagcaagcggtagtcaaccacctccaattccgccaatctcttgcccccatcatttgtctctgacaatacttggtgtttttccctggttttctgtcactggcacaggagggtacagttgggagagtgcgttcctggagctcagtcctgcatttgttcacgtgcctcacagcaggcctttgtgcccttgaatctcaaacatggggatctgcttggtacccagagctctggtcattgtgtccaaccacacaccccaccccatccgtgtcctccatctcacccagaaccacagggtgcccactagtgtcagggcccaaagtgccagccttctcttctgccttacctagtctacctatttatttcctccactttttatcttaaaagtagctaagccatgctggtgcccatactccaagcaggctgcctcagctcagagaagtggtcagagagtagagcacagaacctgtgatgtggggacatttggttttcttgcagatcatttaatgaatcctcaaggactaatgaaataaatgctagactgctg

SEQ ID NO: 6 - protein, 917 aa, Homo sapiens
AARRGCQAASAAAAPGGGGGRESLGRAGLTVGAPGRRPRGAHAMEKLAAGLAGLRWSMGAFPLDLIVSRCRLPTLACLGPGEYAEGVSERDILLIHSCRQWTTVTAHTLEEGHYVIGPKIDIPLQYPGKFKLLEQARDVREPVRYFSSVEEVASVFPDRIFVMEAITFSVKVVSGEFSEDSEVYNFTLHAGDELTLMGQAEILCAKTTKERSRFTTLLRKLGRAGALAGVGGGGPASAGAAGGTGGGGARPVKGKMPCLICMNHRTNESLSLPFQCQGRFSTRSPLELQMQEGEHTVRAIIERVRLPVNVLVPSRPPRNPYDLHPVREGHCYKLVSIISKTVVLGLALRREGPAPLHFLLLTDTPRFALPQGLLAGDPRVERLVRDSASYCRERFDPDEYSTAVREAPAELAEDCASPRRARLCLPAPRAPGLARAPGPLAPAPAGEGDQEYVSPDWAAAPEPAAPPAEIPYEELWAHQGPEGLVRPPPGLDLISFGAAGPPRREPEAPPPPVPPKSEAVKEECRLLNAPPVPPRGGNGSGRLSSSPPVPPRFPKLQPVHSPSSSLSYYSSGLQDGAGSRSGSGSPSPDTYSLYCYPCTWGDCKVGESSSRPAPGPLPSTTQPSQASRALTEPLSGRAASLLGADTPVKTYHSCPPLFKPSHPQKRFAPFGALNPFSGPAYPSGPSAALSSGPRTTSGPVATSGPAYSPGPASPGQAYSAAPPSSCAPSSSSSSEWQEPVLEPFDPFELGQGSSPEPELLRSQEPRAVGTPGPGPRLSPLGPSKAFEPEGLVLHQVPTPLSPAALQGPEAGGALFLTQGRLEGPPASPRDGATGFGVRDASSWQPPADLSALSLEEVSRSLRFIGLSEDVVSFFARERIDGSIFVQLSEDILADDFHLTKLQVKKIMQFIKGWRPKI

SEQ ID NO: 7 - DNA, 44 bp, Artificial Sequence
  synthetic oligonucleotide
gactagttctagatcgcgagcggccgcccttttttttttttttt